(19) United States
(12) Patent Application Publication
(10) Pub. No.: US 2023/0011210 A1
(43) Pub. Date: Jan. 12, 2023

(54) Abrasion Preventing Pad

(71) Applicant: Peach Protex LLC, Miami, FL (US)

(72) Inventor: Haley Alonso, Miami, FL (US)

(21) Appl. No.: 17/859,256

(22) Filed: Jul. 7, 2022

Related U.S. Application Data
(60) Provisional application No. 63/219,119, filed on Jul. 7, 2021.

(51) Int. Cl. A41D 13/05; A41D 13/015; A63B 71/12

(52) U.S. Cl. CPC A41D 13/0575; A41D 13/0525; A41D 13/0156; A63B 71/12

(57) ABSTRACT

A pad may be worn to prevent abrasion or provide a fulcrum during exercise. The pad has a tapering thickness profile to provide support and leverage to the wearer. The pad may have an adhesive side for adhering either to a garment or directly to a wearer. Also disclosed is a variety of garments including pockets in which the pad may be removably retained.

### CROSS-REFERENCE TO RELATED APPLICATIONS

This application claims the benefit of the filing date of U.S. Provisional Patent Application No. 63/219,119 filed Jul. 7, 2021, the disclosure of which is hereby incorporated herein by reference.

### BACKGROUND

Many forms of exercise involve repetitive motion, frequently against a supporting surface or exercise equipment. The repetitive nature of these exercises can cause abrasion to the portion of an individual contacting the supporting surface or equipment. The high-traction types of flooring common in gyms and fitness studios are conducive to such abrasion, but these exercises can result in abrasion to the individual when performed on nearly any surface. For example, sit ups and many other abdominal exercises involve repeated movement while lying down or in a seated position on the floor. Abdominal exercises in particular may thus result in abrasion of an individual's lower back, tailbone area, and seat. Abrasion therefore presents an obstacle to exercise duration and frequency. A way of protecting an individual's skin from a supporting surface would mitigate this obstacle.

### BRIEF SUMMARY

According to one aspect, adhesive pads may be used for releasably adhering directly to a wearer's skin or garment. Such adhesive pads may be worn on their own or in combination with garments having pad-retaining pockets.

According to another aspect, a garment may be provided with a pocket for removably retaining a pad at an area of a wearer's body that contacts a supporting surface or exercise equipment during an exercise. Such pocket may be tailored for a specific shape of pad or shaped to accept one pad of a group of pads of differing shapes. In one example, the garment may be shorts, pants, leggings, underwear and undergarments, or another garment for the lower body, having a pocket placed and dimensioned for removably holding a pad at or near the wearer's tailbone, and possibly extending to the wearer's lower back and/or seat. Alternatively, or in addition, such garment may include a similar pocket for holding a pad at each hip. Garments for the upper body are also contemplated which may have pockets for releasably holding pads at, for example, the wearer's lower, middle, or upper back, elbows, or shoulders.

In either of the above aspects, the pad or pads may be a soft material, possibly with significant shear flexibility, such as, for example, gel, such as silicone gel, or another material having similar mechanical properties. A pad may be shaped to taper from being relatively thin at its outer edges to relatively thick at interior portions remote from the outer edges. A thickest portion of the pad may be, for example, at a center point of the pad. Alternatively, the thickest portion of the pad may located to provide optimal protection, cushioning, and leverage for effective or comfortable performance of a given exercise. At the edges, the pad may taper to being thin enough to be flush with the wearer's skin.

Pads according to the foregoing examples may be available, individually or in kits, in a variety of shapes. A wearer may select a pad or group of pads based on considerations including a type of exercise anticipated, the user's own anatomy, and aesthetic preference, for example. Some pads may have a number of individually flexible extensions or tabs extending from a central hub, thus enabling the pad overall with greater flexibility and protecting certain remote portions of a wearer's skin with less material than relatively simple shapes like circles or entirely convex polygons. Pads may also be shaped and configured for individual use or use in combination with another pad, and an accompanying garment may accordingly have either a pocket large enough to hold multiple cooperating pads or a group of pockets each being configured to hold one pad of a group of cooperating pads.

### BRIEF DESCRIPTION OF THE DRAWINGS

FIGS. 1A-1C illustrate a wearer of a pad according to the present disclosure sitting up from a supine position.

FIG. 2A illustrates a height-wise thickness profile of the pad on a vertical-dorsal plane.

FIG. 2B illustrates a width-wise thickness profile of the pad on a lateral-dorsal plane.

FIG. 2C illustrates a width-wise thickness profile of the pad according to another arrangement on a lateral-dorsal plane.

FIG. 2D illustrates a width-wise thickness profile of a pair of the pads according to another arrangement on a lateral-dorsal plane.

FIGS. 3A-3Q illustrate possible designs for broad sides of the pad according to various arrangements.

FIGS. 4A-4D illustrate various lower body garments including pockets for receiving a pad.

FIG. 5A is a view of an outer side of a pad according to another arrangement.

FIG. 5B is an oblique perspective view of a of the pad of FIG. 5A.

FIG. 5C is a view of a wearer facing side of the pad of FIG. 5A.

FIG. 5D is a cross-section on plane 5D-5D of FIG. 5C.

FIG. 5E is a lateral view of the pad of FIG. 5A.

### DETAILED DESCRIPTION

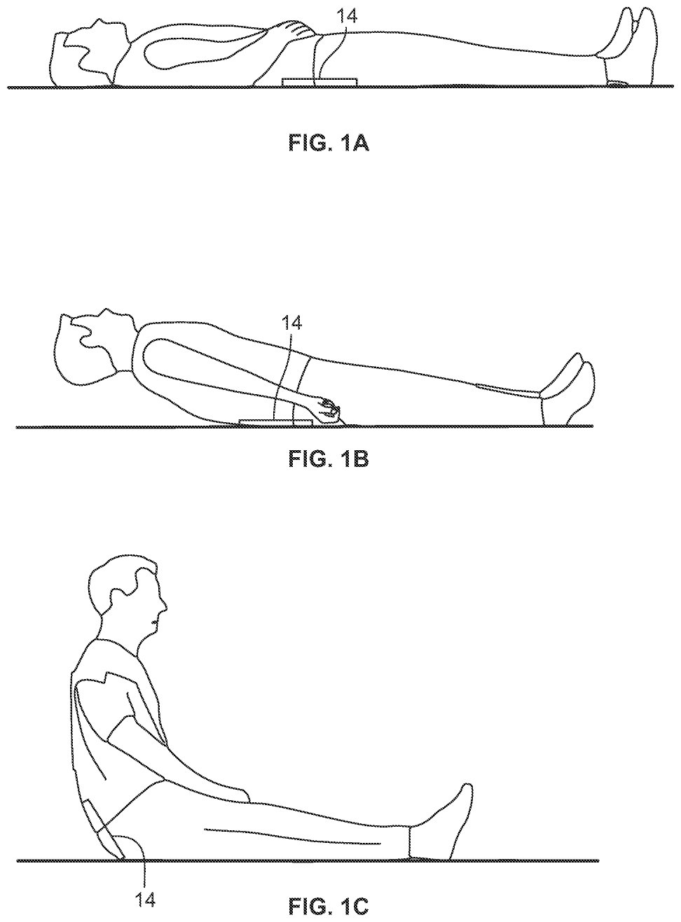

A pad 14, as shown on a wearer in FIGS. 1A-1C, according to the present example is shaped and sized to prevent abrasion of the wearer's lower back and seat while enacting sit ups. The pad 14 may be worn in a location such that nearly an entire side of the pad would press against a planar floor if the wearer lies on the floor with a flat back as illustrated in FIG. 1A. The pad 14 may be adhered to the wearer's skin or clothes by an adhesive substance or surface on one side of the pad 14. Alternatively or in addition, the pad 14 may be placed inside a pocket in a garment as will be discussed in more detail below. In the illustrated example, the pad 14 is worn such that the area of the pad 14 pressed against the ground will decrease as the user sits up as illustrated in FIG. 1B until, for example, only a third or less of the pad remains pressed between the wearer's seat and the floor when the wearer reaches a sitting position as illustrated in FIG. 1C. Though not illustrated, pads may instead, or in addition, be worn in other locations, such as to cover each hip, thus providing support and preventing abrasion if the wearer enacts side-lying exercises.

The pad 14 may be a soft material, possibly with significant shear flexibility, such as, for example, gel, such as silicone gel, or another material having similar mechanical properties. Other suitable materials include fabric or foam. The pad 14 may have a different cover material, such as an exterior layer of fabric.

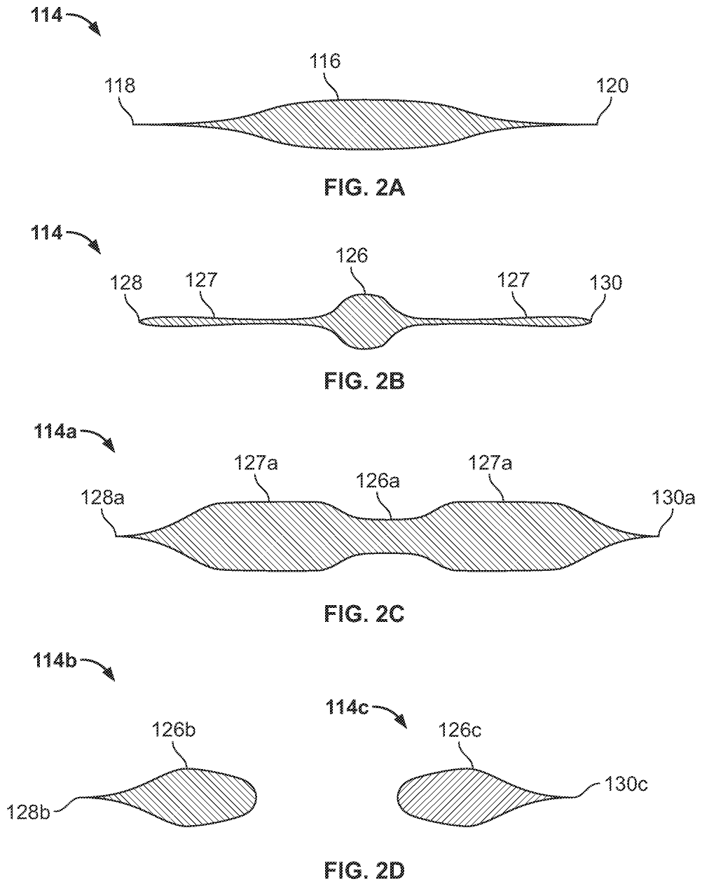

Pads 14 according to any of the foregoing examples may be a pad 114 having a variable thickness across its breadth as illustrated in FIGS. 2A-2D. FIG. 2A illustrates a cross-section of the pad 114 on a vertical-dorsal plane extending through a thickest point of the pad 114 providing an example of a height-wise thickness profile of the pad. A vertical location 116 of a thickest point of the pad 114 of the example illustrated in FIG. 2A is halfway between an upper edge 118 and lower edge 120 of the pad 114. The upper end of the pad 114 is the upper edge 118 on the vertical-dorsal cross-section of the pad 114 that extends highest, and the lower end of the pad 114 is the lower edge 120 on the vertical-dorsal cross-section of the pad 114 that extends lowest. In the example described here, FIG. 2A is assumed to represent a cross-section of the pad 114 that extends both highest and lowest such that the upper edge 118 is the upper end of the pad 114 and the lower edge 120 is the lower end of the pad 114.

The vertical location 116 of the thickest point may be higher or lower in other arrangements so as to provide better protection or leverage for exercise according to a wearer's anatomy and preference. Example alternative locations for the vertical location 116 of the thickest point include equal to or about one third of the way from the upper edge 118, or upper end, to the lower edge 120, or lower end, or equal to or about one third of the way from the lower edge 120 to the upper edge 118. In further alternatives, there may be thickest portions of the pad 114 at two different vertical locations, each of which may be any of the above described placements for the vertical location 116 of the thickest point. Regardless of the location and number of the thickest portion of the pad 114, the pad tapers gradually in thickness therefrom to being relatively thin near the upper edge 118 and lower edge 120 of the pad 114. The pad 114 may taper such that the upper edge 118 and lower edge 120 would be flush with the wearer's skin when the pad 114 is worn on the body.

The height-wise thickness profile illustrated in FIG. 2A, or any of the above described variations thereon, may hold true across a portion of a width of the pad. That is, while the actual thickness of the pad at a given vertical location may differ across a width of the pad, the thickness proportions of the height-wise thickness profile of the pad may remain generally consistent with what is illustrated in FIG. 2A or described above with regard to variations thereto across, for example, a quarter or more, a third or more, half or more, two thirds or more, or three quarters or more of a width of the pad.

The pad 114 may be contoured such that a cross-section of the pad 114 on most lateral-dorsal planes along the height of the pad would generally resemble the vertical-dorsal cross-section illustrated in FIG. 2A. Throughout this disclosure, the terms “vertical” and “dorsal” or “ventral,” when used with respect to any pad discussed herein, refers to directions relative to the body of a standing individual wearing the pad in an intended orientation, and lateral refers to horizontal directions that are perpendicular to the dorsal and ventral directions. However, various other contours are possible. For example, FIGS. 2B and 2C both show lateral or width-wise thickness profiles including two lateral wings with thicknesses that stay generally constant across a lateral extent thereof on either side of a narrow portion of differing thickness.

As shown in FIG. 2B, the pad 114 may be of relatively or generally constant thickness across most of a width defined between a first lateral edge 128 and an opposite, second lateral edge 130, both lateral edges tapering narrow enough in the illustrated example to lie flush on a wearer's skin, with a pronounced increase in thickness in a relatively narrow area immediately adjacent a lateral location 126 of a thickest point of the pad 114. The left end of the pad 114 is the first lateral edge 128 on the lateral-dorsal cross-section of the pad 114 that extends furthest left, and the right end of the pad 114 is the second lateral edge 130 on the vertical-dorsal cross-section of the pad 114 that extends furthest right. In the example described here, FIG. 2B is assumed to represent a cross-section of the pad 114 that extends both furthest left and furthest right such that the first lateral edge 128 is the left end of the pad 114 and the second lateral edge 128 is the right end of the pad 114.

The lateral location 126 of the thickest location of the pad 114 is halfway between the first lateral edge 128 and the second lateral edge 130 of the pad 114. Thus, for a pad with most lateral-dorsal cross-sections generally resembling the lateral-dorsal cross-section illustrated in FIG. 2B, the thickest portion of the pad 114 forms a vertical rib that may extend between the wearer's spinal erectors or buttocks to support the spine. Wings 127 on either side of the rib each have constant, or nearly constant, thickness across most of their respective lateral extent, but may taper and become thinner at their lateral edges.

A lateral-dorsal cross-section of an alternative arrangement of the pad 114a is illustrated in FIG. 2C, which may have a height-wise thickness profile such as that illustrated in FIG. 2A or described above with regard to pad 114. In particular contrast to the example of FIG. 2B, the pad 114a of FIG. 2C has thicker wings 127a on opposite sides of a vertically extending thin neck 126a. The wings 127a extend between a first lateral edge 128a and second lateral edge 130a, both lateral edges tapering to be thin enough to lie flush on a wearer's skin. The pad 114a thus provides more support off-center from the wearer's spine, which some wearers may prefer for certain exercises. The vertical-dorsal cross-sections of the pad 114a at each wing 127a of a thickest point of the pad 114a may taper away from a vertical location of the respective thickest point in the same manner as described above with regard to pad 114. Similarly, the vertical location of the thickest portion of the pad 114a may be halfway between upper and lower edges of the pad 114a, equal to or about one third of the way from the upper edge of the pad 114a to the lower edge of the pad 114a, or equal to or about one third of the way from the lower edge of the pad 114a to the upper edge of the pad 114a. Thus, the fulcrum that the pad 114a provides for a wearer performing, for example, sit ups may be approximately in the middle of the pad 114a, or higher or lower on the wearer's body than the middle of the pad 114a.

As noted above, FIGS. 2B and 2C both show lateral or width-wise thickness profiles including two lateral wings of constant or relatively constant thickness on either side of a vertically extending centerline of a differing thickness. In one example, not necessarily consistent with FIGS. 2B and 2C, a width-wise thickness profile at a given vertical location on a pad may be such that 80% or more of the pad at that vertical location has a substantially constant thickness, while a lateral or width-wise center point of the pad at that vertical location has a substantially different thickness. Here, “substantially constant” means within 10% or less of a given thickness, or from 90% and 110% of that thickness, while “substantially different” means more than 10% different from the given thickness. Variations to the foregoing quantities are also contemplated. The area of substantially constant thickness, which need not be continuous, may extend across, for example, a total of at least 60%, at least 75%, at least 85%, or at least 90% of a width of the pad at a given vertical location. The areas of substantially constant thickness on the wings may stay within 15% or within 5% of a given thickness, or may be entirely equal in thickness. Whether the wings or the center portion of the pad are thicker, the thicker portion may be, for example, 50% or more thicker, 75% or more thicker, or 100% or more thicker than the thinner portion. All combinations of the foregoing numerical variations are contemplated as applicable to the thickness profiles of both FIGS. 2B and 2C. The thicknesses are to be measured when the pad is at rest, i.e., without acute stress on any point causing the pad to deform, such as when the pad lays flat on a planar surface.

In another alternative, two symmetrical pads 114b, 114c as shown in FIG. 2D may be used together instead of the above described pads 14, 114, 114a. The two pads 114b, 114c may have a height-wise thickness profile such as that illustrated in FIG. 2A or described above with regard to pad 114. In such example, a first lateral edge 128b on one pad 114b and an opposite, second lateral edge 130c on the other pad 114c may be placed at any of the example locations for the lateral edges of any of the above described pads 14, 114, 114a. Both lateral edges 128b, 130c may taper thin enough to lie flush on a wearer's skin. Moreover, a corresponding garment may be designed with two pockets designed to hold the pads 114b, 114c at such locations.

The pads 114b, 114c each have a respective lateral location 126b, 126c of a thickest portion thereof. The thicknesses of the pads 114b, 114c may taper along the height or width thereof in any of the manners described above with regard to the pads 114, 114a of FIGS. 2A-2C. Thus, similar to the pad 114a of FIG. 2C, the pads 114b, 114c of FIG. 2D provide support off-center from the wearer's spine, which some wearers may prefer for certain exercises.

The pads 114b, 114c may, in contrast to the illustrated example, be substantially flat, and thus when spaced either according to the placement of two respective pockets in a garment or according to an intended placement upon a wearer, may create to the numerical proportions described above as examples with regard to FIGS. 2B and 2C. In other words, at least one vertical location, each of the pads 114b, 114c may provide one of the above described wings with substantially constant thickness across a respective flat width thereof, with the respective lateral edges 128b, 130c tapering to be flush with the wearer's skin. When the pads 114b, 114c are placed in respective pockets in a garment, or upon a user, the sum of the flat widths may be at least 60%, at least 75%, at least 80% at least 85%, or at least 90% of a distance between the first lateral edge 128b and the second lateral edge 130c. A center point of the configuration of the pads 114b, 114c would therefore be in an unpadded space between the pads 114b, 114c.

The proportions of the lateral or width-wise thickness profiles illustrated in FIGS. 2B-2D, or any of the above described variations thereof, may remain true across various vertical extents of pads according to this disclosure. That is, for a given pad, the actual thickness of the center portion and wings may change continuously across a vertical extent of the pad while the proportions of the thicknesses for each lateral-dorsal cross-section within a portion of the pad will adhere to one of the above described mathematical relationships or one of the examples illustrated in FIGS. 2B-2D. For example, one quarter or more, 30% or more, one third or more, 40% or more, half or more, 60% or more, two thirds or more, 70% or more, three quarters or more, 80% or more, or 90% or more of a vertical extent of a pad may adhere to any of the above described mathematical relationships of wing thickness to centerline thickness or the width-wise thickness profiles illustrated in any of FIGS. 2B-2D.

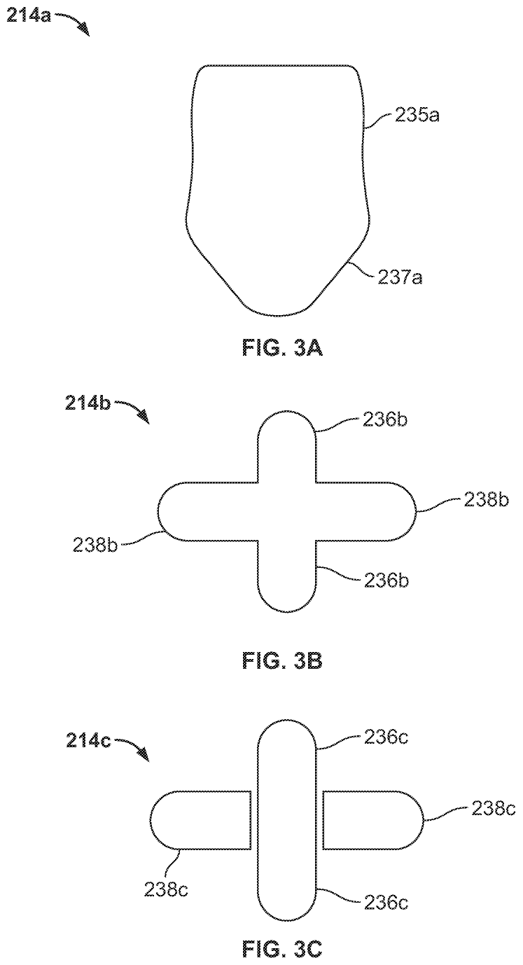

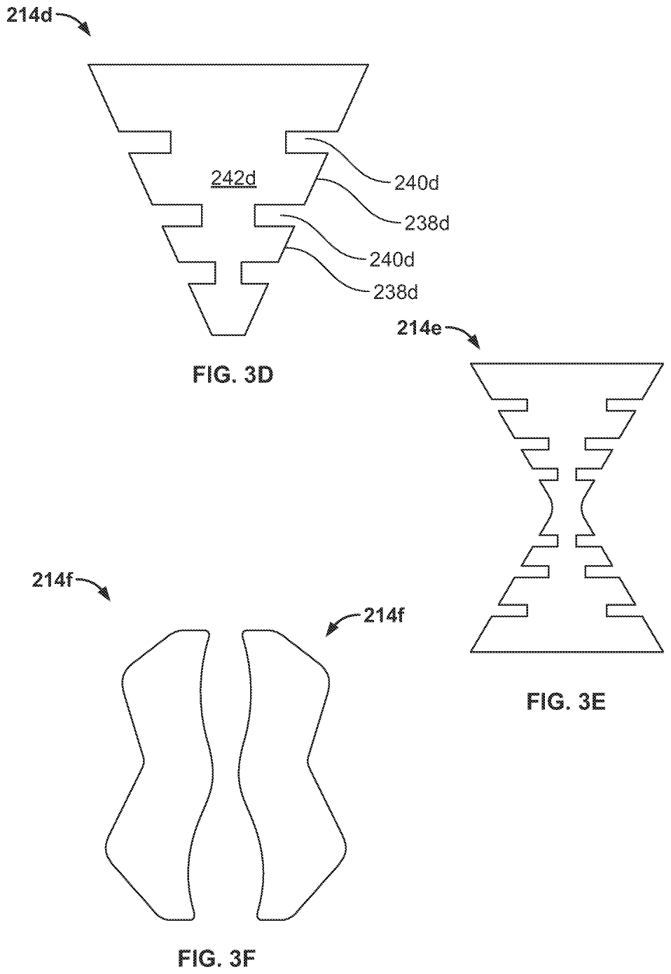

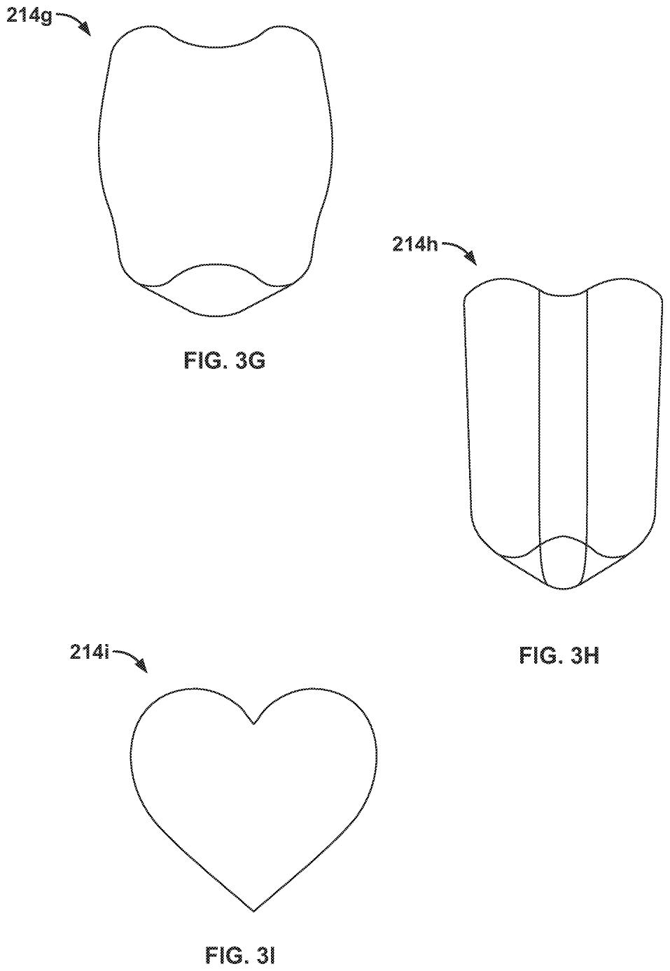

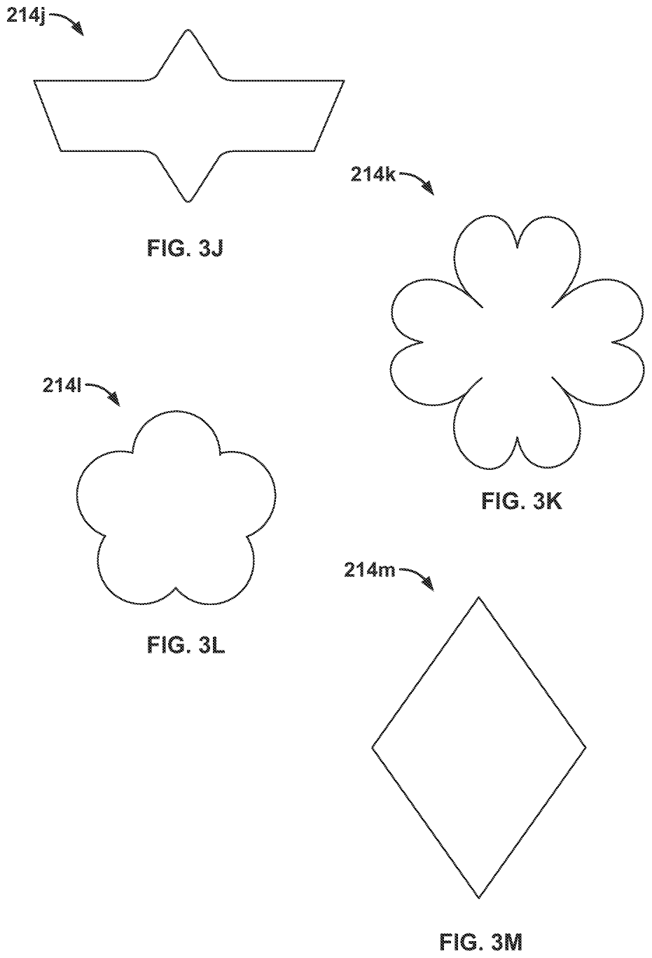

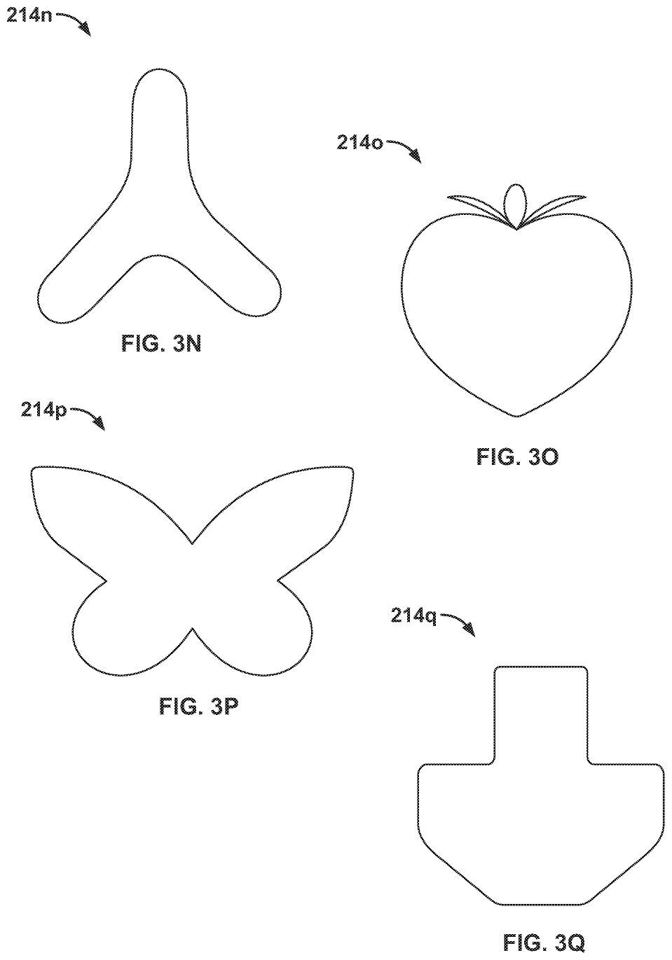

FIGS. 3A-3Q each illustrate a broad side design for any of the above described pads, meaning that the perspective of the viewer of each of FIGS. 3A-3Q is aligned with the thickness direction as described above with regard to FIGS. 2A-2D. That is, because the pads of the present disclosure are smaller in front-to-back thickness than in width and height, viewing any of the pads of the present disclosure from a lateral perspective or a vertical perspective would show the viewer a narrow side of the pad, whereas viewing any of the pads of the present disclosure from a front or back perspective would show a broad side of the pad. Any of the pad designs of FIGS. 3A-3Q may therefore be included with a garment including a pocket of corresponding shape or may be provided with an adhesive on one or both sides for adhering the pad either directly to a wearer or onto clothing. Each pad design in any of FIGS. 3A-3Q may be implemented with any of the thickness profiles described above with regard to FIGS. 2A-2D, though certain combinations of thickness profile and pad design are described below. Moreover, certain merely exemplary orientations are stated with regard to some designs below or suggested with directional terms such as “upper,” “lower,” and “lateral,” but each of the below pad designs may be worn in any orientation or location upon a wearer, and garments may be provided with pockets to hold the pad of any design in any orientation or location. For each pad design of FIGS. 3A-3Q, an example orientation upon a standing wearer with up, down, left, and right corresponding to the illustrated orientation is specifically contemplated.

FIG. 3A illustrates a pad 214a with a polygonal shape. The polygonal shape of the pad 214a includes two longer sides forming lateral edges of an upper portion 235a. In the illustrated example, the lateral edges of the upper portion 235a of the pad 214a are inclined slightly toward one another, such that the upper portion 235a narrows with increasing proximity to an upper edge of the pad 214a. However, in other examples, the lateral edges of the upper portion 235a may instead be parallel to one another, thus giving the pad 214a a shape similar to that of a baseball plate. The triangular lower end 237a of the pad 214a may be positioned to generally follow the triangular shape of the sacrum, while the upper portion 235a extends up to the lumbar portion of the wearer's back.

The pad 214b of FIG. 3B generally resembles a plus sign, with four arms 236b, 238b extending in four cardinal directions and meeting at a center point. The two vertically extending arms 236b, which are equal in length to one another, are shorter than the two laterally extending arms 238b, which are also equal in length to one another, in the illustrated example. However, in alternative examples, the vertically extending arms 236b may have length equal to or greater than that of the laterally extending arms 238b. The vertically extending arms 236b run along the spine while the laterally extending arms 238b may be positioned to cushion the ilia and act as fulcra for abdominal exercises. The construction of the pad 214b with multiple independent arms 236b, 238b enables the wearer to adjust the arms 236b, 238b independently according to the wearer's anatomy and preference. With regard to the pad of FIG. 3B, the independence of the arms 236b, 236c refers to the ability of one arm to bend or stretch to some degree while one or more of the other arms may remain stationary or bend or stretch in a different direction. The independence of the arms 236b, 238b also reduces transfer of strain on one part of the pad 214b to another such that a wearer may be able to bend and twist across a relatively wide range of motion without dislodging the pad 214b.

The pad 214c of FIG. 3C is generally similar to the pad 214b in all respects and possible variations except that the laterally extending arms 238c are separably connectable to the vertically extending arms 236c. The laterally extending arms 238c may be connectable to the vertically extending arms 236c by reversible fastenings, such as, for example, hook-and-loop patches or straps, buttons, clasps, or buckles. In other examples, the laterally extending arms 238c may simply be separate from the vertically extending arms 236c, without connecting elements. The vertically extending arms 236c together thus form a single bar-shaped pad while the two laterally extending arms 238c are each a separate, smaller bar-shaped pad. The separation of the laterally extending arms 238c gives the wearer even greater freedom to bend and twist without dislodging any of the arms 236c, 238c. Also, depending on the shape of a pocket intended to receive a pad, inserting the multiple portions forming the pad design 214c of FIG. 3C one at a time may be easier than inserting the entirety of the pad 214b of FIG. 3B. The two laterally extending arms 238c could first be guided into corresponding extensions of a plus-shaped pocket, followed by the bar provided by the two vertically extending arms 236c. The same steps could be executed in reverse to remove the pad 214c.

FIG. 3D illustrates a pad 214d has a triangular overall shape. However, the overall shape of the pad 214d includes horizontally extending arms 238d separated from one another by notches 240d. Similar to the pads 214b, 214c described above, the division of the pad 214d by notches 240d enables the arms 238d to move independently from one another, thus allowing the wearer to bend and twist further without dislodging the pad 214d. Any other pad design described herein may be modified with similar notches to increase the overall flexibility of the pad in a similar manner. Notches may also be made in the upper and lower edges of any pad in addition or in alternative to notches on the lateral edges. The arms 238d may be thinner than a middle portion 242d of the pad 214d into which the notches 240d do not extend. Though not shown in the illustrated example, the notches 240d may be bridged by, for example, webbing or ribs extending between the arms 238d. The webbing, ribs, or other features extending between the arms 238d may be elastic. The number of notches 240d and arms 238d illustrated is merely an example, and more or fewer notches 240d and arms 238d may exist on any side of the pad 214d according to other examples.

FIG. 3E illustrates a pad 214e similar to the pad 214d of FIG. 3D, except that the pad 214e is formed by two triangles meeting at a point to define an overall hourglass shape. The pad 214e is thus able to twist at its middle with relative freedom.

FIG. 3F illustrates a pair of pads 214f that may be used together. The pads 214f are a mirror image of one another. The pads 214f are particularly suited to the thickness profile of FIG. 2D or any of the variations thereon described above. If the pads 214f are provided with a corresponding pocketed garment, the garment may include one pocket for retaining both pads 214f or two pockets each for retaining a respective one of the pads 214f. In the illustrated example, the pads 214f are shaped generally like opposed butterfly wings or each as a respective half of an overall hourglass shape, but paired pads according to other examples may be generally straight, arcuate, or any of a variety of other shapes.

FIGS. 3G and 3H respectively illustrate pads 214g and 214h. The pads 214g and 214h both have a chevron shape, but differ in thickness profile. The pad 214g is generally flat with a pronounced increase in thickness near a lateral centerline according to the thickness profile of FIG. 2B. The pad 214h, by contrast, is relatively thin along its lateral centerline, with thicker wings on each lateral side, according to the thickness profile of FIG. 2C. The pads 214g and 214h thus provide differences in support that users may choose based on personal preference and a planned type of exercise.

The above described functional differences between the foregoing pads 214a-214h are also applicable to pad designs with aesthetic aspects in ways that may be readily appreciated upon observation. Further design examples include a heart 214i shown in FIG. 31, a pad 214j in the shape of a diamond centered and superimposed on a trapezoid such that the diamond extends vertically beyond the trapezoid and the mutually non-parallel edges of the trapezoid extend laterally beyond the diamond as shown in FIG. 3J, a four leaf clover 214k as shown in FIG. 3K, a flower shape 214l as shown in FIG. 3L, a diamond 214m as shown in FIG. 3M, a shape 214n with three arms extending at 120° angles relative to one another as shown in FIG. 3N, a peach 214o as shown in FIG. 3O, butterfly wings 214p as shown in FIG. 3P, and a shape 214q with a rectangle extending from a widest edge of a hexagon, said widest edge being located between two mutually parallel edges of the hexagon as shown in FIG. 3Q. Pads 214k and 214l each include notches therein providing some additional flexibility in a manner similar to pads 214d, 214e. Pad 214n has independently movable arms similar to the arms 236b, 238b of pad 214b. The term “independent” is used with regard to pad 214n in the same sense as it was used above with regard to pad 214b. Pads 214i, 214j, 214m, 214o, and 214q have points or triangular shapes at their lower end and may therefore be placed to lie over the sacrum and protect the ilia in a similar manner to pad 214a, while pads 214j and 214q in particular may provide additional support to the small of the back. Pad 214p may have the thickness profile of either FIG. 2B or FIG. 2C to provide more support on either the spine or the obliques similar to pads 214g or 214h, respectively.

Women's and men's garments of various types may be provided with one or more pockets each for retaining a pad, permanently or removably, for protecting a wearer from abrasion during exercise. The pad retainable by the garment may be any of the above described pads, though optionally the pad may lack any adhesive. Further, the location and dimensions of the pockets described for any garment described herein correspond to locations on a wearer's body if the garment is assumed to be form-fitting, and any of the above described pads may be sized and worn adhesively so as to be coextensive with any of the locations on a wearer's body corresponding to the extent of any garment pocket described herein.

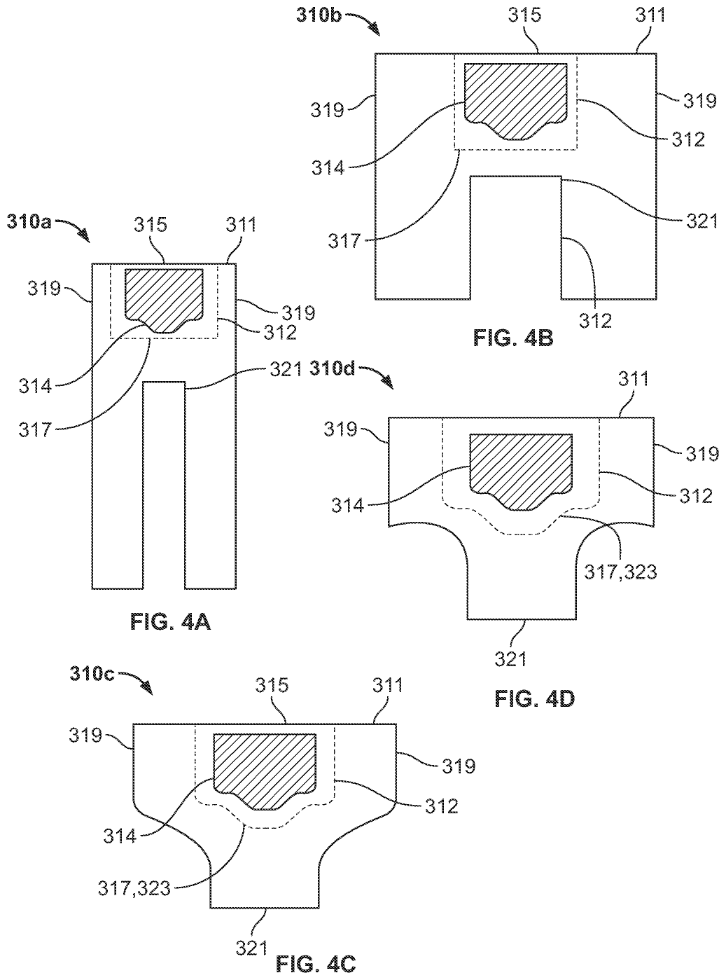

Examples of lower body garments, each with one pocket for removably holding a pad generally over a wearer's sacrum and tailbone, include pants or leggings 310a as shown in FIG. 4A, which may be either loose or form fitting, shorts or boxers 310b as shown in FIG. 4B, and varieties of undergarments 310c, 310d as shown in FIGS. 4C and 4D. The illustrated garments are merely exemplary, and pockets for retaining pads may be integrated into any known type of garment according to the principles of the present disclosure. Other examples of lower body garments in which such pockets may be integrated include other kinds of underwear, any variety of pants, and skorts.

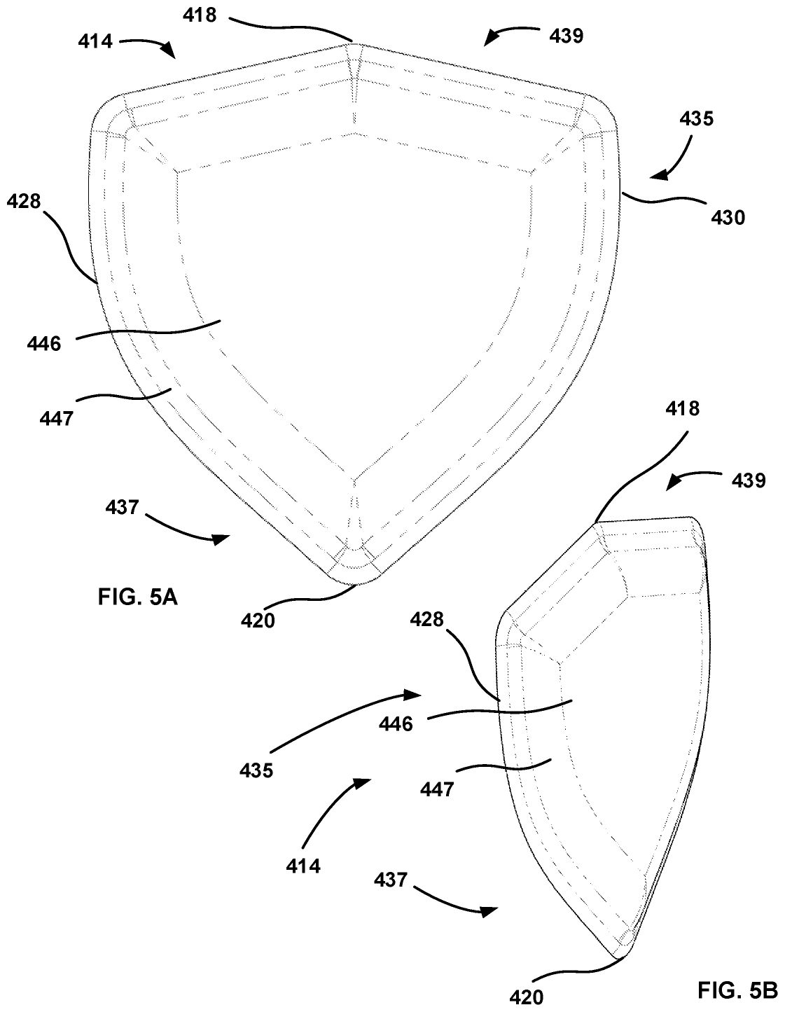

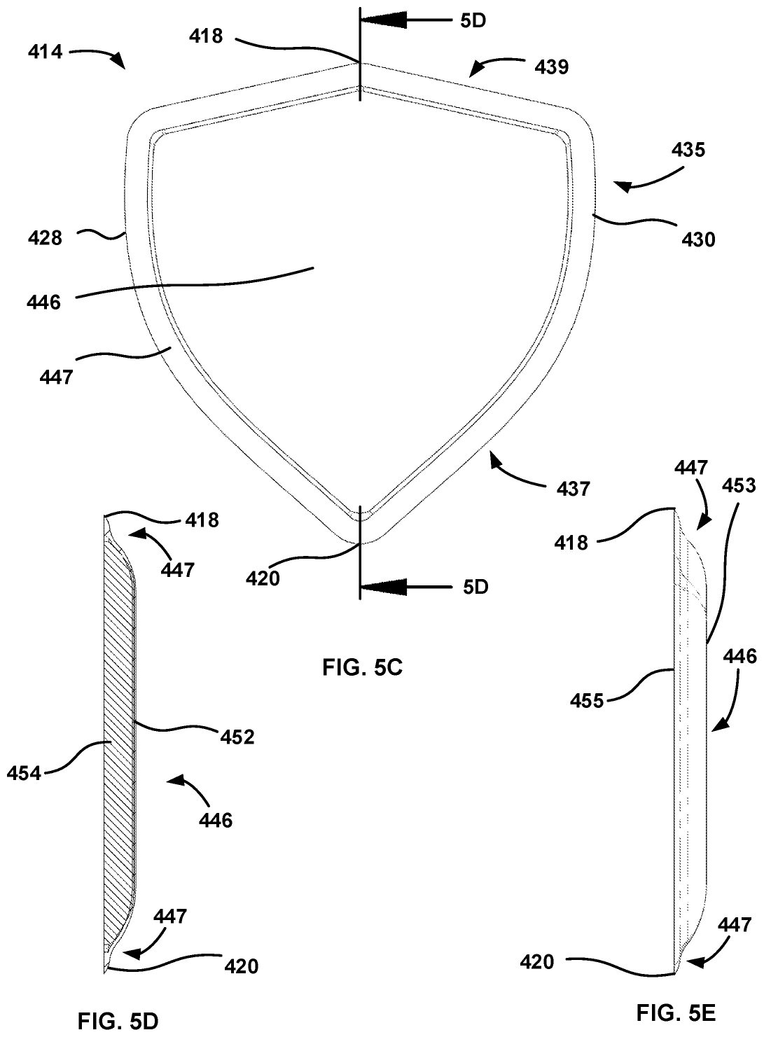

Each such garment includes a pocket 312 shaped and sized to hold a pad 314. Each pad 314 illustrated in FIGS. 4A-4D is merely exemplary, and in other examples a pad having any of the shapes illustrated or described above with regard to FIGS. 1A-3Q or below with regard to FIGS. 5A-5E may be used in the pockets 312. The pockets 312 may be shaped to closely fit any such pad.

The pocket 312 of the illustrated example is shown open at a top edge thereof, meaning near the waistband 311 of the garments of FIGS. 4A-4D. In other embodiments, pocket 312 may be at other locations of the garment. In further alternative embodiments, pocket 312 may have no opening, and may therefore permanently retain any pad disposed therein. The opening of the pocket 312 may also have a closure such as hook and loop strips, buttons, or a zipper to retain the pad 314 in place. Further, a side of the pocket 312 opposite from the opening may have a non-linear profile 323 shaped to match and conform to a portion of an outer perimeter the pad 314 to guide the pad 314 into a specific position, or the pocket 312 may be generally round or rectangular in shape so as to accommodate any of a variety of pads 314 of different shapes. Such non-linear profile 323 is specifically shown in FIGS. 4C and 4D, but may be used in a pocket 312 of any garment.

The location and the dimension of the pocket 312 may be described in terms of its position between features of the garment 310, with vertical, lateral, and dorsal or ventral directions being relative to the orientation of the garment 310. For this purpose, the terms “inseam,” “outseam,” and “waistband” as used throughout this disclosure refer to the locations where those features are found on typical garments for the lower body, and may thus indicate the same locations on, for example, garments where such seams are absent or atypically located. However, in each instance, positions relative to an actual waistband, inseam, and outseam are also contemplated. For example, on a pair of pants or leggings 310a of a standard waistline (i.e., neither high-rise nor low-rise) an upper edge 315 of the pocket 314 may be located on or at an upper edge of a waistband 311 or separated from the top of the waistband 311 by, for example, 10% or less, 20% or less, or 25% or less of a vertical distance between the top of the waistband 311 and the top of the inseam 321. A lower edge 317 of the pocket 314 may be separated from the top of the waistband 311 by equal to or about half, or between 40% and 60%, of the vertical distance between the top of the waistband 311 and the top of the inseam 321. The pocket 312 is centered on a back side of the leggings 310a between the outseams 319, and the lateral edges at a laterally widest point of the pocket 314 may be spaced such that the pocket 312 extends across half, two thirds, or between half and two thirds of a lateral distance between the outseams 319. The edges of the pocket 314 of the other garments 310b, 310c, 310d, or any other garment for the lower body wherein such a pocket may be implemented, may be at the corresponding locations on such garments. A pocket 314 in a high-rise garment would have the same proportions relative to the waistband 311, inseam 321, and outseams 319, except that the lower edge 317 thereof could be located anywhere from halfway to three-quarters of the vertical distance from the upper edge of the waistband 311 to the upper end of the inseam 321, in addition to the possible locations relative to the waistband 311 and inseam 321 for the lower edge 317 described above with regard to standard waistline leggings 310a.

In examples wherein the pad 314 is configured for use with a garment, such as any of the above garments 310a, 310b, 310c, 310d, the pad 314 fits snugly within the pocket 312, and the upper, lower, and lateral ends of the pad 314 while in the pocket 312 therefore extend to the above described locations of the upper edge 315, lower edge 317, and lateral edges of the pocket 312.

As noted above with regard to FIGS. 1A-3Q, a pad may be configured for use without a garment having a pocket 312 as described above. Such adhesive pads 14 may be provided in various sizes so that the upper, lower, and lateral ends of the pad 14 would extend to the locations on a typical pair of leggings of common size corresponding to the above described locations for upper edge 315, lower edge 317, and lateral edges, respectively, of the pocket 312 described above for the leggings 310a. Similarly, if the leggings 310a are assumed to be form fitting, the above described locations for the upper edge 315, lower edge 317, and lateral edges of the pocket 312 of the leggings 310a correspond to locations on a wearer's anatomy where the upper, lower, and lateral ends, respectively, of an adhesive pad 14 may be sized to lie when applied directly to a wearer's skin. However, adhesive pads may alternatively be larger than the above described dimensions for the pocket 312. For example, adhesive pads may be sized to extend up to the narrowest portion of the wearer's waist.

Like the pads of FIGS. 3A-3Q, FIG. 5A illustrates a broad side design for a pad 414, meaning that the perspective of the viewer of FIG. 5A is aligned with the thickness direction as described above with regard to FIGS. 2A-2D. The pad design of FIGS. 5A-5E may be included with a garment including a pocket of corresponding shape or may be provided with an adhesive on one or both sides for adhering the pad either directly to a wearer or onto clothing. The pad design in FIGS. 5A-5E may be implemented with any of the thickness profiles described above with regard to FIGS. 2A-2C, though FIGS. 5D and 5E illustrate a specific thickness profile. Moreover, certain merely exemplary orientations are stated with regard to the design of FIGS. 5A-5E or suggested with directional terms such as “upper,” “lower,” and “lateral,” but each of the below pad designs may be worn in any orientation or location upon a wearer, and garments may be provided with pockets to hold the pad of any design in any orientation or location. For the pad design of FIGS. 5A-5E, an example orientation upon a standing wearer with up, down, left, and right corresponding to the illustrated orientation is specifically contemplated.

The pad 414 of FIGS. 5A-5E has a body portion 446 of constant, or at least substantially constant, thickness. Thus, the body portion 446 is a thickest portion of the pad 414 and has constant thickness may be of a given thickness across its width and height, whereas a body portion 446 may be within 10% of a given thickness across its width and height. Like the pad of FIGS. 2A-2C, the pad has an upper edge 418, a lower edge 420, a first lateral edge 428, and a second lateral edge 430. The edges 418, 420, 428, and 430 are each part of a periphery 447 of the pad that tapers in an S-curve shape from the thickness of the body portion 446 to nothing, as can be seen at the top and bottom of FIGS. 5D and 5E. The way that the cross-sectional shape of the pad 414 slopes away from the relatively thick body portion 446 to the thin periphery 447 allows the wearer to easily and comfortably rock onto and off of the pad 414. The periphery 447 also increases the proportion of the surface area available to be adhered to the user relative to the size of the cushion provided by the body portion. In the illustrated example, the periphery 447 extends a constant distance away from the body portion 446 around an entire perimeter of the body portion 446 so that the body portion 446 has the same shape as the pad 414 overall. In other examples, the periphery 447 may extend away from the body portion 446 by a distance that varies around the perimeter of the body portion 446. Moreover, though the periphery 447 is illustrated as tapering to nothing on an S-curve, in other examples the periphery 447 may taper to nothing on a profile having any other shape.

The broad side design of the pad 414 of FIGS. 5A-5E is roughly diamond or shield shaped, having a widest portion 435 defined between the two lateral edges 428, 430 of the pad 414. The widest portion 435 is defined vertically between an upper triangle 439 and a lower triangle 437. The upper triangle 439 comes to a point defining the upper edge 418 of the pad 414 and the lower triangle 437 comes to a point defining the lower edge 420 of the pad 414. In one example, the pad 414 may be worn so that the upper edge sits on the small of the wearer's back while the lower edge 420 is positioned over the end of the wearer's tailbone.

The body portion 446 may extend across, for example, at least 60%, at least 75%, at least 85%, or at least 90% of a total width of the pad at the vertical location of the widest portion 435. Similarly, the body portion 446 may extend across, for example, at least 60%, at least 75%, at least 85%, or at least 90% of a total height of the pad 414 defined as the distance between the upper edge 418 and the lower edge 420.

The pad 414 may have a wearer facing surface 455 and an outer surface 453, as shown in FIG. 5E. As shown in FIG. 5D, the wearer facing surface 455 may be provided by a core material 454, while the outer surface 453 and much of the periphery 447 may be provided by a coating material 452. In some examples, the core material 453 may be a molded silicone gel that both acts as a cushion and naturally adheres to the wearer's skin, while the coating material 452 may be a fabric material, such as a type of fabric used in fabric bandages. In other examples, the coating material 452 may be omitted. In still further examples, the user facing surface 455 may be provided by a material, which may optionally be the coating material 452, other than the core material 453. In addition, the core material 453 and coating material 452 may differ from foregoing examples. Moreover, an adhesive material other than the core material 453 may be applied to the wearer facing surface 455 regardless of whether the core material 453 or the coating material 452 defines the wearer facing surface 455. It is noted that the foregoing details and potential alternatives described above with regard to the shape and construction of the pad 414 of FIGS. 5A-5E, including the arrangement of layers, the materials, the thickness profile, the S-curve periphery, and the proportion of the periphery to the rest of the pad can be applied to pads having any of the broad side shapes shown in any of the other figures in the present application or described anywhere else in the present specification.

Although the concepts herein have been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A pad for protecting a wearer's skin during exercise, the pad including:
two opposed broad sides extending across a height defined between first and second ends of the pad, and a width defined perpendicular to the height and between third and fourth ends of the pad, and a thickness of the pad defined between the two opposed broad sides tapers to nothing at an opposed pair of the ends from a thickest portion of the pad that is spaced from both ends of the opposed pair of the ends.

2. The pad of claim 1, wherein the thickness of the pad follows:
a height-wise thickness profile defined between the broad sides, wherein at least one lateral location on the pad tapers from a thickest height-wise location spaced from the first end and the second end to relatively thin points at the first end and the second end; and
a width-wise thickness profile defined between the broad sides, wherein at least 75% of the width of the pad being within 10% of a given thickness, while a center point of the width-wise thickness profile differs from the given thickness by at least 10%, holds true for at least one quarter of a height-wise extent of the pad.

3. The pad of claim 2, wherein a thickness of the pad at the center point of the width-wise thickness profile is at least twice the given thickness.

4. The pad of claim 1, wherein a thickest point of the pad is centered between the first end and the second end.

5. The pad of claim 1, wherein a thickest point of the pad is at a location separated from the first end of the pad by twice as great a distance as from the second end of the pad.

6. The pad of claim 1, including a middle portion spaced form at least two of the ends and multiple arms extending from the middle portion.

7. The pad of claim 6, wherein the middle portion is thicker than the arms.

8. The pad of claim 1, including a triangular portion extending to a point defining a lower end of the pad.

9. The pad of claim 1, having an adhesive layer on at least one of the broad sides.

10. An apparel kit including:
the pad of claim 1; and
a garment including a pocket sized and shaped to removably receive the pad.

11. The kit of claim 10, wherein the pocket includes an opening and an edge opposite the opening, and the edge opposite of the opening has a non-linear profile matching a portion of an outer perimeter of the pad.

12. The kit of claim 10, wherein the garment is a garment for the lower body having a waistband location and a top of inseam location, and the pocket is located on a back side of the garment between the top of inseam location and waistband location.

13. An apparel kit including:
the pad of claim 1, wherein the pad is a first pad; and
a second pad shaped to be a mirror image of the first pad.

14. The pad of claim 1, wherein the thickest portion of the pad extends across at least 60% of the width and the height of the pad.

15. The pad of claim 14, wherein a periphery defining a remainder of the pad extends around a perimeter of the thickest portion of the pad and tapers away to nothing on an S-curve profile.

16. Apparel for the lower body comprising:
a garment for the lower body including a pocket, a waistband location, and a top of inseam location, the pocket being located on a back side of the garment between the waistband location and the top of inseam location; and
a pad including:
two opposed broad sides extending across a height, the height being defined between first and second ends of the pad, and a width, the width being defined perpendicular to the height and between third and fourth edges of the pad; wherein
a height-wise thickness profile defined between the broad sides, wherein at least one lateral location on the pad tapers from a thickest height-wise location spaced from the first end and the second end to relatively thin points at the first end and the second end.

17. The apparel of claim 16, wherein the pocket includes a lower edge, an upper edge, and two opposed lateral edges, and wherein the lower edge of the pocket is located at a position separated from the waistband location by at least two thirds of a distance between the waistband location and the top of inseam location, and the upper edge of the pocket is located at a position separated from the waistband location by a distance that is less than 10% of the distance between the waistband location and the top of inseam location; and
the pad is sized and shaped to fit snugly between the two lateral edges of the pocket and against an entire width of the lower edge of the pocket.

18. The apparel of claim 17, wherein the pocket is shaped and sized such that, when the pad is disposed within the pocket snugly against the lower edge and both lateral edges of the pocket, an entire side of the pad would press against a planar floor if a wearer of the garment would lie on the floor with a flat back, an area of the pad pressed against the floor would decrease as the wearer sits up, and half or less of the pad would remain pressed between the wearer and the floor when the wearer reaches a sitting position.

19. The apparel of claim 16, wherein a width-wise thickness profile defined between the broad sides includes at least 75% of the width of the pad being within 10% of a given thickness, while a center point of the width-wise thickness profile differs from the given thickness by at least 10%, holds true for at least one quarter of a height-wise extent of the pad.

20. The apparel of claim 16, wherein:
the pocket is a first pocket;
the pad is a first pad;
the garment includes a second pocket which is a mirror image of the first pocket; and
the apparel further includes a second pad which is a mirror image of the first pad.
